(19) United States
(12) Patent Application Publication
(10) Pub. No.: US 2003/0217419 A1
(43) Pub. Date: Nov. 27, 2003

(54) Cosmetic use of at least one hydrophobin for treating keratin materials, and compositions used

(75) Inventor: Gabin Vic, Compiegne (FR)

Correspondence Address: STEPTOE & JOHNSON LLP, 1330 Connecticut Ave., NW, Washington, DC, 20036, US

(73) Assignee: L'Oreal, Paris (FR)

(21) Appl. No.: 10/318,383

(22) Filed: Dec. 13, 2002

(30) Foreign Application Priority Data
Dec. 14, 2001 (FR) 0116250

(51) Int. Cl. A61K 7/06; A61K 7/11; A61K 7/13

(52) U.S. Cl. 8/406; 424/70.13; 424/70.14

(57) ABSTRACT

The present invention relates to the use of a cosmetic composition for treating keratin materials, comprising at least one hydrophobin, and also to processes for cosmetically treating keratin materials.

The present invention relates to the cosmetic use for treating keratin materials of at least one hydrophobin, and also to compositions and processes for cosmetically treating the skin and/or the hair.

Cosmetic products intended for treating the hair, that provide the hair with properties such as styling, sheen and disentangling, mainly use polymers that are not readily adsorbed onto the hair or that are adsorbed well but give a heavy, generally sticky feel. Furthermore, the cosmetic effects observed are not long-lasting since the polymers are rapidly removed on shampooing.

The Applicant has discovered, surprisingly, that hydrophobins can be used to treat the surface of keratin materials in order to obtain a cosmetic deposit that withstands several shampoo washes.

One subject of the invention is thus the cosmetic use for treating keratin materials of a composition containing at least one hydrophobin.

Another subject of the invention is the cosmetic compositions used.

Another subject of the invention is a process for cosmetically treating the skin and/or the hair using hydrophobin.

Other subjects of the present patent application will emerge on reading the description and the examples that follow.

The cosmetic use that constitutes a first subject of the invention comprises the use for treating keratin materials, in particular the hair, of a composition containing at least one hydrophobin in a cosmetically acceptable medium.

The hydrophobins used in accordance with the invention are natural hydrophobins obtained by extraction, hydrophobins obtained by synthesis or hydrophobins modified by chemical reaction by methods known to those skilled in the art (Bioconjugate Techniques, Academic Press 1996).

Among the modified hydrophobins that may be mentioned are hydrophobins bearing cosmetic active agents such as dyes, sunscreens, polymers and bactericides. These agents are attached by grafting and formation of a covalent bond. These modifications may also involve the replacement, omission or incorporation of one or more amino acids, with the proviso of maintaining at least the characteristic adsorption property of the hydrophobins, these products being able to be obtained by genetic engineering.

The hydrophobins may also be covalently attached to supports such as mineral or organic particles.

The hydrophobins used according to the invention are small proteins of about 100 amino acids secreted by filamentous fungi, which fulfil a large number of functions in the growth and development of these fungi.

These proteins appear to be ubiquitous among filamentous fungi. More than 34 genes encoding different hydrophobins have been isolated from 16 species of fungi. Among these fungi, ascomycetes and basidiomycetes are particularly preferred.

In the primary structure of these proteins, 8 cysteine residues, four of which are grouped into two pairs, are found in each molecule in a characteristic order and with a characteristic spacing defined by the following sequence:

Xₙ-C-X_{5 to 9}-C-C-X_{11 to 39}-C-X_{2 to 23}-C-X_{5 to 9}-C-C-X_{6 to 18}-C-Xₘ

X representing any amino acid, C representing a cysteine residue, n and m denoting integers, the other figures indicating the number of intercalating amino acids (for example 5 to 9).

Depending on their solubility after self-assembly on a surface, the hydrophobins may be separated into two classes: class I and class II. The characteristics of these two classes of hydrophobins are described in the article by H. A. B. Wösten and M. L. de Vocht (Biochimica et Biophysica Acta, (2000), vol. 1469 pp. 79-86).

An example of a class II hydrophobin that may be mentioned is the hydrophobin from cerato-ulmine or from cryparine. Class I hydrophobins, and among these SC3 or SC3P, will preferably be used.

SC3 is a glycosylated hydrophobin of 112 amino acids obtained from the fungus Schizophyllum commune.

The compositions used according to the invention preferably comprise from 10 ppb to 20% and in particular from 1 ppm to 10%, on a weight-for-weight basis, of at least one hydrophobin relative to the total weight of the composition.

Even more preferably, the compositions comprise from 5 ppm to 5% by weight of at least one hydrophobin relative to the total weight of the composition.

The compositions of the present invention may contain, in addition to hydrophobin, at least one polysaccharide and/or at least one cosmetic active agent. The polysaccharide may be extracted from the culture medium of the fungus producing it, together with the hydrophobin, or isolated separately and added subsequently. It may also be obtained from another natural or synthetic strain.

Preferably, the polysaccharide used in the composition according to the invention is schizophyllan.

Schizophyllan is a simple branched glucan, in which the repeating unit consists of three β 1-3 linked D-glucose molecules, one of them being linked to a D-glucose molecule via a β 1-6 linkage.

When the fungus Schizophyllum commune is grown, in addition to the hydrophobin SC3, schizophyllan is secreted into the medium.

The compositions according to the invention preferably comprise from 10 ppb to 20% by weight of at least one polysaccharide relative to the total weight of the composition.

More preferably, the compositions comprise from 1 ppm to 10% by weight of at least one polysaccharide relative to the total weight of the composition.

Even more preferably, the compositions comprise from 5 ppm to 5% by weight of at least one polysaccharide relative to the total weight of the composition.

The cosmetic compositions according to the invention may also comprise hydrophobin mixed with one or more cosmetic active agents.

The cosmetic active agent(s) included in the compositions according to the invention may be chosen from the group composed of natural or synthetic soluble polymers, natural or synthetic insoluble polymers, pigments, moisturizers, antidandruff agents, plant, animal or synthetic oils, animal or plant waxes such as ceramides, proteins, enzymes, mineral, metallic or organic particles, vitamins, sunscreens, dyes, fragrances and antioxidants.

The concentrations of these cosmetic active agents are individually between 0.01 and 90% of the total weight of the composition.

The compositions of the invention may also comprise surfactants in the same proportions. The surfactants and polymers may be of nonionic, cationic, anionic or amphoteric nature. Preserving agents may also be added thereto.

In the cosmetic compositions according to the invention, the cosmetically acceptable medium consists of water or a mixture of water and solvent. This solvent may be a C1-C4 lower alcohol, for instance ethanol or isopropanol or a polyol or a polyol ether, for instance glycerol, polypropylene glycol or ethers thereof.

The compositions according to the invention contain between 0 and 50% of one or more solvents.

The compositions in accordance with the invention may be in the form of an aqueous, alcoholic or aqueous-alcoholic solution, a lotion, a dispersion, a fluid or thick cream, a gel, a stick, an emulsion or a mousse. They may optionally be packaged in an aerosol device.

Another subject of the invention is a process for cosmetically treating the skin and/or the hair, which consists in applying a sufficient amount of a composition according to the invention to the keratin materials, at a temperature of between 10 and 100° C., this application optionally being followed by rinsing and/or drying at room temperature or under heat.

For the purposes of the present invention, the expression “keratin material” means the skin, head hair, the eyelashes, the nails and other hairs. Preferably, the compositions according to the invention are applied to head hair.

One variant consists in applying the composition containing at least one hydrophobin and then in following this application by a treatment based on a surfactant composition at room temperature or under heat.

Before applying the cosmetic composition according to the invention, a composition containing either a reducing agent or an oxidizing agent for permanently reshaping the hair, an oxidation dye composition, a bleaching composition, a shampoo or a styling composition may be applied to the hair.

The hair treatment process according to the invention may also consist in applying the cosmetic composition according to the invention after treating the hair fibre with a cosmetic active agent.

The example that follows is intended to illustrate a composition that may be used according to the invention:

| Compound | Amount | |
|---|---|---|
| Hydrophobin SC3 from | 0.001 | gram |
| Schizophyllum commune | | |
| Schizophyllan | 0.001 | gram |
| Phosphate buffer at pH 7 | 0.5 | mol/l |
| Demineralized water | qs. 100 | grams |

1. Cosmetic use for treating keratin materials of a composition containing at least one hydrophobin in a cosmetically acceptable medium.

2. Use according to claim 1, characterized in that the protein from the hydrophobin class is a class II hydrophobin.

3. Use according to claim 1, characterized in that the protein from the hydrophobin class is a class I hydrophobin.

4. Use according to any one of claims 1 to 3, characterized in that the hydrophobin is a hydrophobin modified chemically or by incorporation, replacement or omission of one or more amino acids.

5. Use according to any one of claims 1 to 4, characterized in that the hydrophobin is a hydrophobin modified by genetic engineering.

6. Use according to any one of claims 1 to 5, characterized in that the hydrophobin is obtained by extraction.

7. Use according to claim 3, characterized in that the protein from class I of the hydrophobins is hydrophobin SC3 or SC3P.

8. Use according to claims 3 and 6, characterized in that the hydrophobin SC3 or SC3P is produced by the fungus Schizophyllum commune.

9. Use according to any one of claims 1 to 8, characterized in that the composition contains from 10 ppb to 20% by weight of at least one hydrophobin as defined in any one of claims 1 to 8, relative to the total weight of the composition.

10. Use according to claim 9, characterized in that the composition contains from 1 ppm to 10% by weight of at least one hydrophobin as defined in any one of claims 1 to 8, relative to the total weight of the composition.

11. Use according to claim 10, characterized in that the composition contains from 5 ppm to 5% by weight of at least one hydrophobin as defined in any one of claims 1 to 8, relative to the total weight of the composition.

12. Cosmetic composition, characterized in that the hydrophobin is a chemically modified hydrophobin.

13. Cosmetic composition, characterized in that it comprises, in a cosmetically acceptable medium, at least one hydrophobin and at least one polysaccharide.

14. Composition according to claim 13, characterized in that the polysaccharide is schizophyllan produced by the fungus Schizophyllum commune.

15. Composition according to either of claims 13 and 14, characterized in that it contains from 1 ppb to 20% by weight of at least one polysaccharide relative to the total weight of the composition.

16. Composition according to claim 15, characterized in that it contains from 1 ppm to 10% by weight of at least one polysaccharide relative to the total weight of the composition.

17. Composition according to claim 16, characterized in that it contains from 5 ppm to 5% by weight of at least one polysaccharide relative to the total weight of the composition.

18. Composition according to any one of claims 12 to 17, characterized in that it comprises at least one hydrophobin and at least one cosmetic active agent other than polysaccharides.

19. Composition according to claim 18, characterized in that the cosmetic active agent(s) may be chosen from the group composed of natural or synthetic soluble polymers, natural or synthetic insoluble polymers, pigments, moisturizers, antidandruff agents, plant, animal or synthetic oils, animal or plant waxes, proteins, enzymes, mineral, metallic or organic particles, vitamins, sunscreens, dyes, fragrances, preserving agents and antioxidants.

20. Composition according to any one of claims 12 to 19, characterized in that the cosmetically acceptable medium consists of water or a mixture of water and solvent.

21. Composition according to any one of claims 12 to 20, characterized in that it is in the form of an aqueous, alcoholic or aqueous-alcoholic solution, a lotion, a dispersion, a thick fluid cream, a gel, a stick, an emulsion or a mousse.

22. Process for cosmetically treating keratin materials, characterized in that it involves the use of a composition as defined in any one of claims 1 to 11.

23. Process according to claim 22, characterized in that a sufficient amount of a composition as defined in any one of claims 12 to 21 is applied to the keratin materials, at a temperature of between 10 and 100° C., and this application is optionally followed by rinsing and/or drying at room temperature or under heat.

24. Process according to claim 22 or 23, characterized in that the application of the composition with hydrophobin(s) is followed by the application of a composition containing at least one surfactant.

25. Process according to claim 24, characterized in that a sufficient amount of the cosmetic composition containing at least hydrophobin is applied to the hair after pretreatment of the hair fibre with a cosmetic active agent.

26. Process according to claim 25, characterized in that a composition containing either a reducing agent or an oxidizing agent for permanently reshaping the hair, an oxidation dye composition, a bleaching composition, a shampoo or a styling composition is applied to the hair, and then a sufficient amount of the cosmetic composition containing at least hydrophobin is applied.
